(19) United States
(12) Patent Application Publication
(10) Pub. No.: US 2014/0093952 A1
(43) Pub. Date: Apr. 3, 2014

(54) Bioreactor Tangential Flow Perfusion Filter System

(71) Applicant: David Serway, Rockville, MD (US)

(72) Inventor: David Serway, Rockville, MD (US)

(21) Appl. No.: 13/633,272

(22) Filed: Oct. 2, 2012

(51) Int. Cl. C12M 1/12

(52) U.S. Cl. 435/297.4; 435/297.2

(57) ABSTRACT

A bioreactor hollow fiber perfusion system increases the capacity of standard fed batch bioreactors. The bioreactor hollow fiber perfusion system cycles bioreactor mass through a hollow fiber tangential flow filter which separates the metabolic wastes (as well as proteins) from the biomass material allowing the reactions in the bioreactor to continue when compared to a fed batch bioreactor. The bioreactor hollow fiber perfusion system preferably includes a low shear gamma stable disposable pumphead responsible for biomass re-cycling and can be easy installed or replaced without the risk of contamination.

### BACKGROUND OF THE INVENTION

The present invention relates to bioreactors and in particular to an improved bioreactor filtering system and method including a tangential flow filter.

Bioreactors provide an environment supporting biological activity. Various forms of bioreactors exist and some forms require filtering biological material in the bioreactor during the biological process. Pump and filter systems are used for such filtering and often require a sterile environment. Unfortunately, when pumps or filters of known systems require service or replacement, the required procedures can be time consuming due to the requirement to maintain the sterile environment.

Further, all known bioreactor systems build up metabolic waste in the bioreactor which limits the amplification or cell growth within the reactor. As a result known high capacity bioreactor systems require very large and expensive reactors.

### BRIEF SUMMARY OF THE INVENTION

The present invention addresses the above and other needs by providing a bioreactor hollow fiber perfusion system which increases the capacity of standard fed batch bioreactors. The bioreactor filtering system recycles bioreactor fluids, including cells, through a lumen in a hollow fiber of a tangential flow filter. The tangential flow filter includes larger pore membranes (>0.1 uM), which remove metabolic wastes (and expressed proteins). Removing the waste allows the continued proliferation of cells within the bioreactor, to continually expressed recombinant proteins, antibodies or other extracellular components that are of interest. Hollow fiber membranes with pore sizes <0.1 u can be utilized to generate a bioreactor environment which retains products of interest while passing metabolic wastes through the hollow fiber tangential flow filter.

In accordance with one aspect of the invention, there is provided a tangential flow filter system including a disposable low shear pump. The disposable low shear pump allows recycling of the cells without contamination or damage to the cells. The disposable low shear recirculating pump and associated tubing is aseptically connectable to the bioreactor.

In accordance with another aspect of the invention, there is provided a tangential flow filter system including either manual or automatic control of the perfusion process for operation. Some modes of operation are designed for seed reactors, continuous perfusion reactors, concentrated fed batch perfusion as well as cell or cell debris clarification (post transfection or Cell Lysis).

In accordance with still another aspect of the invention, there is provided a disposable perfusion tangential flow filtration system which decreases existing bioreactors size requirements. Systems one tenth the size of known fed-batch processing systems can provide protein productivity equivalent or better extracellular proteins as well as overall concentration of material.

In accordance with yet another aspect of the invention, there is provided a disposable perfusion tangential flow filtration system which facilitates implementing or changing a pre-assembled, pre-sterile perfusion tangential flow processing system without impacting the bioreactor sterility both during the operation or upon start up completely eliminates the need for autoclaving components when utilizing the disposable perfusion tangential flow filtration system. The system is designed to connect to either disposable, glass and even stainless steel reactors. The complete perfusion systems includes the disposable pumphead, hollow fiber module and associated connections which is designed to be gentle on cells or other biological material without impacting viability and is scalable.

In accordance with still another aspect of the invention, there is provided a hollow fiber perfusion tangential flow filtration system providing quick assembly to a bioreactor processing flow path Module (hollow fiber), Bag and Tubing (MBT) assembly. The MBT assembly includes a bag containing media feeding the reactor or a permeate bag collecting metabolic wastes. In some cases the bioreactor vessel may be a bag. A pre-sterilized processing MBT assembly includes low shear a re-circulation pump, automatic control of filtration sequences of operation including: the operation of seed reactors; continuous tangential flow; concentrated cell tangential flow; concentrated fed-batch tangential flow; as well as cell clarification (post transfection or cell lysis). The hollow fiber perfusion tangential flow filtration system decreases required volume within bioreactors to obtain similar or better productivity to a bioreactor which is ten times larger or greater when compared to fed batch processing. Higher cell productivity with improved protein productivity is obtained. The perfusion system flow path provides a simple procedure for changing of the processing loop without impacting sterility. The disposable MBT assembly eliminates the need for autoclaving components and is designed to connect to either disposable reactors, re-usable glass and stainless steel reactors. The MBT assembly is pre-sterilized with a disposable low shear pumphead which is gentle on cells without impacting viability and is scaleable to obtain the same performance as a system having very large reactor vessels using tangential flow, maintains equivalent or even better protein productivity when compared to known fed-batch processing, provides an easily changed tangential flow processing loop without impacting sterility, eliminates a need for autoclaving components, is designed to connect to either disposable reactors, glass, and stainless steel reactors, and includes a disposable pumphead which is gentle on cells without impacting viability and scalable.

### BRIEF DESCRIPTION OF THE SEVERAL VIEWS OF THE DRAWING

The above and other aspects, features and advantages of the present invention will be more apparent from the following more particular description thereof, presented in conjunction with the following drawings wherein:

FIG. 1 is a bioreactor system according to the present invention.

FIG. 2 is a tangential flow perfusion filtering system according to the present invention.

FIG. 3A shows a detailed view of a first tangential flow perfusion filtering system according to the present invention.

FIG. 3B shows a detailed view of a second tangential flow perfusion filtering system according to the present invention.

FIG. 3C shows a detailed view of a third tangential flow perfusion filtering system according to the present invention.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing one or more preferred embodiments of the invention. The scope of the invention should be determined with reference to the claims.

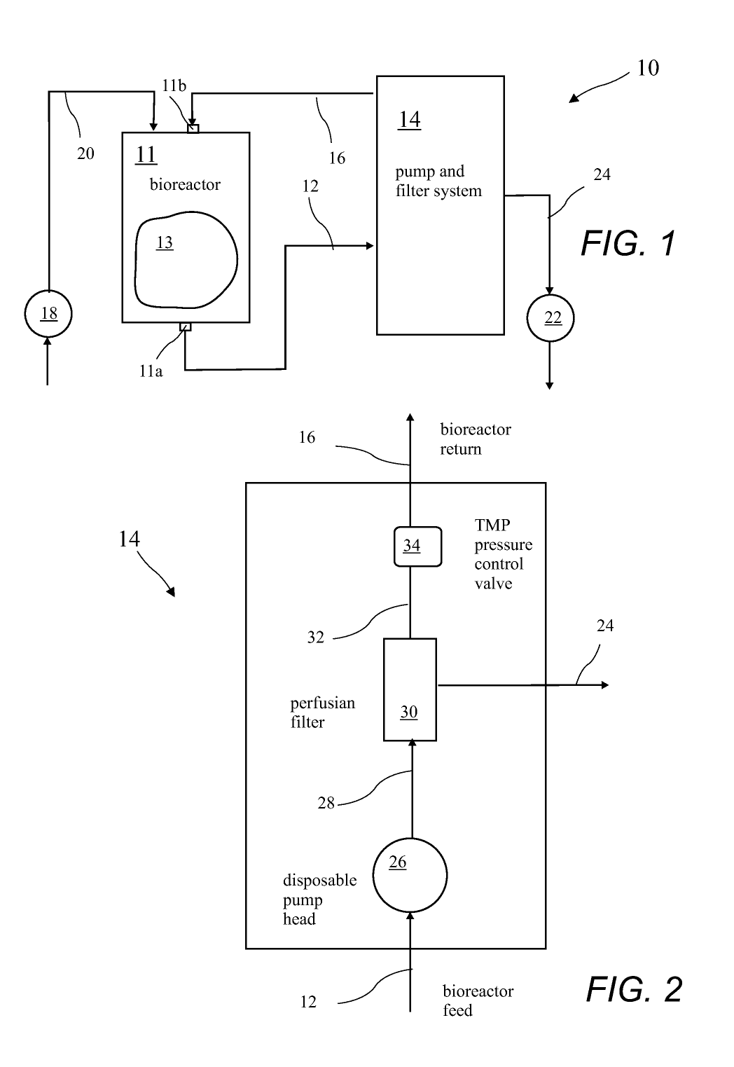

A bioreactor system 10 according to the present invention is shown in FIG. 1. The bioreactor system 10 includes a bioreactor vessel 11 containing bioreactor fluid 13 and a pre-sterilized tangential flow perfusion filtering system 14. The pre-sterilized tangential flow perfusion filtering system 14 is connected between a bioreactor outlet 11a and bioreactor inlet 11b to receive a bioreactor material flow 12 from the bioreactor 11 and return a filter flow 16 to the bioreactor 11. The bioreactor system 10 cycles bioreactor fluid through the tangential flow perfusion filtering system 14 which separates out metabolic wastes and/or protein waste material and thereby allows the reaction in the bioreactor vessel 11 to continue to remove to completion allowing higher cell densities within the same bioreactor which provides greater proteins to be expressed due to this increased density of viable cells.

The bioreactor vessel 11 receives a re-circulation of bio-mass flow 20 through a pre-sterilized assembly consisting of pump, hollow fiber and associated fittings and connections 18. The pump preferably includes a low shear gamma stable disposable levitating pumphead, for example, a model number MPD-200 low shear re-circulation pump manufactured by Levitronix in Waltham, Massachusetts. The MDP-200 includes a magnetically levitated rotor inside a disposable pumphead, and stator windings in the pump body, allowing simple removal and replacement of the pumphead. A bioreactor material flow 12 passes from the bioreactor vessel 11 to the tangential flow perfusion filtering system 14 and a hollow fiber filtered flow 16 passes from the tangential flow perfusion filtering system 14 back to the bioreactor vessel 11. A metabolic waste material flow 24 is stripped from the bioreactor contained volume 12 by the hollow fiber perfusion filtering system 14. The metabolic waste, as well as associated proteins, are drawing from the hollow fiber perfusion tangential flow system 14 by a permeate pump 22.

The tangential flow perfusion filtering system 14 according to the present invention is shown in FIG. 2. The tangential flow perfusion filtering system 14 includes a tangential flow filter pump 26, which includes the disposable pumphead which simplifies initial set up and maintenance. The pump 26 re-circulates the bioreactor material flow 12 from the bioreactor vessel 11 and advances the bioreactor material flow 12 through a hollow fiber tangential flow filter 30. A non-invasive transmembrane pressure control valve 34 in line with the hollow fiber flow 16 from the hollow fiber tangential flow filter 30 to the bioreactor vessel 11, controls the pressure within the hollow fiber tangential flow filter 30. Bioreactor waste material 24 is continually removed from the bioreactor cell mass which flows through the lumen of the hollow fiber 12 and is drawn through the hollow fiber filter 30 by the permeate pump 22. The pump 26 and tangential flow filter waste pump 22 are controlled by software to maintain the desired flow through the hollow fiber tangential flow filter 30.

A detailed view of a first tangential flow perfusion filtering system 14a according to the present invention is shown in FIG. 2a. The tangential flow perfusion filtering system 14a receives the bioreactor material flow 12 through a sanitary connection 38 to the bioreactor 40 connected to a female connector 40. The flow 12 passes through a non-invasive ultrasonic flow meter and then through the disposable pump 26 to provide a controlled pump biomass flow 12a through the tangential flow filter 30 through a sanitary connection 50 that is connected to the return line from the hollow fiber tangential flow filter 30. The non-invasive TMP pressure control valve located on the return flow 16 back to the bioreactor can be used to maintain the correct pressure within the hollow fiber tangential flow filter 30. The return flow 16 passes through an aseptic connection 40/38 and returns to the bioreactor vessel 11.

Pressure sensors 44 and 48 reside in communication with the flows 12a and 16 respectively before and after the hollow fiber tangential flow filter 30. The fittings 50 include nipples for attachment of the pressure sensors 44 and 48. The hollow fiber tangential flow filter 30 includes ports 30a and 30b connected to the metabolic waste material flow 24 for the release of waste material separated from the flow 12a. Pressure sensor 49 resides in communication with the return flow 16.

The hollow fiber tangential flow filter 30 is preferably a hollow fiber filter which can be either a microporous or ultrafilter pore size. The hollow fiber tangential flow filter 30 is pre-sterilized with the associated sensors and connections, and manufactured with no biocides, and only animal free glycerine is present within the pores of the hollow fiber tangential flow filter 30. The hollow fiber tangential flow filter 30 eliminates the need for autoclaving prior to using. Preferably, a very low protein binding chemistry is used, however, polysulfone (PS) as well as other chemistries can be utilized. Preferably, a very low protein binding chemistry Modified Polyether Sulfone (mPES) membrane is used. The perfusion hollow fiber can be either a 0.5 mm lumen or 1.0 mm lumen with scaleable hollow fiber elements to accommodate varying bioreactor sizes. An example of an acceptable hollow fiber tangential flow filter 30 is a hollow fiber filter such as the KrosFlo Filter Module manufactured by Spectrum Labs in Rancho Dominguez, Calif.

The pump 26 preferably includes a disposable pumphead. The time to set-up, flush and sterilize a perfusion system which is not completely disposable is extensive in comparison. Components in the pump 56 include parts which in some instances require replacement between each run. Mechanical components which wear or tear and may give off debris into the bioreactor filter. A preferred disposable pumphead includes no mechanical interaction between parts, and a preferred pumphead includes magnet elements which rotate in the presence of a rotating magnetic field, effectively as a rotor in an electric motor. An example of a preferred pump is made by Levitronix in Switzerland (Zurick) with offices in Waltham, Mass.

The valve 34 is preferably a non-invasive valve which resides outside tubing carrying the return flow 16. The valve “squeezes” the tubing to restrict and control the flow. Such a valve 34 is non-invasive and provides a commercial advantage since the return line to the reactor is situated thru the valve to regulate the applied pressure on the membrane.

The pump 26 and filter 22 in the filtering system 14a are preferably connected by flexible tubing allowing easy changing of the elements. Such tubing allows aseptically replacement of the filter 22 during a run in case the hollow fiber pore becomes plugged, over-loaded with material which therefore provides easy exchange to a new perfusion hollow fiber assembly.

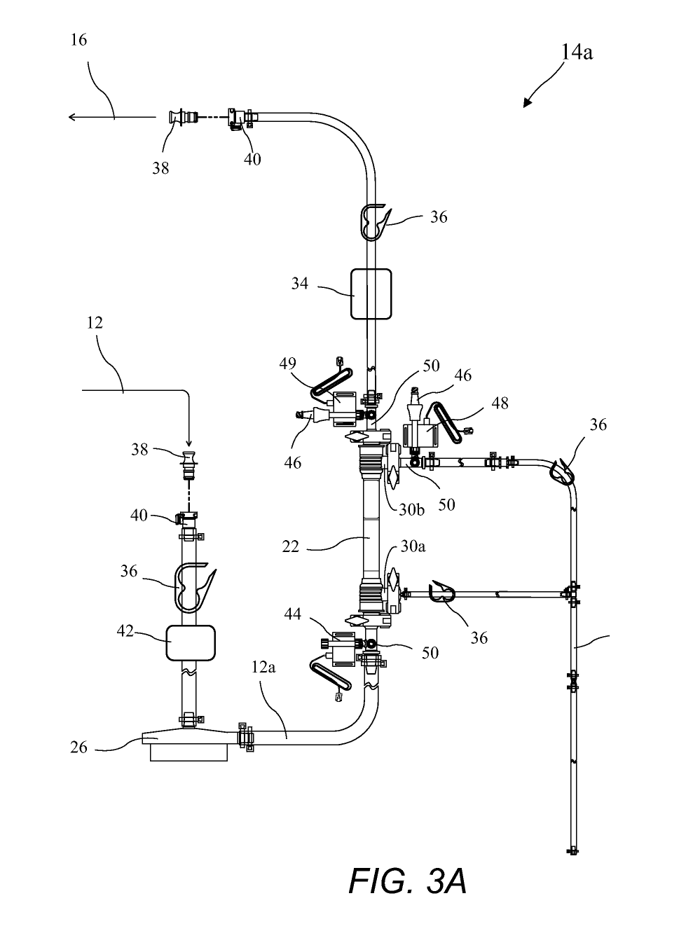

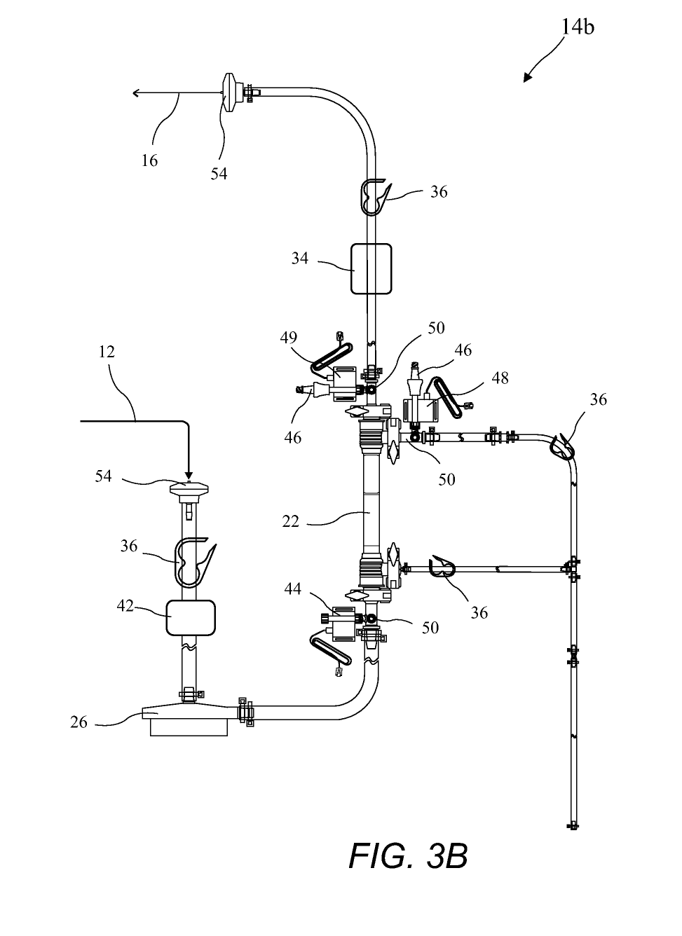

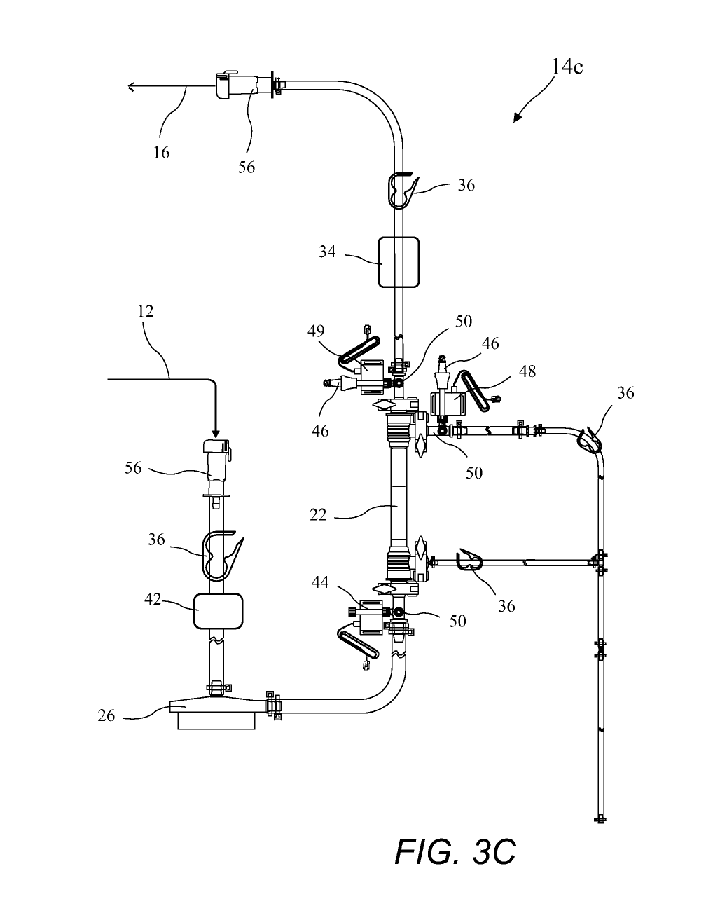

A second tangential flow perfusion filtering system 14b is shown in FIG. 3B. The filtering system 14b replaces the connectors 38 and 40 with a first disposable aseptic connector 54. Filter systems according to the present invention may further include tube welding or aseptic connectors manufactured by GE, Pall, Millipores and other, and filtering systems according to the present invention including any aseptic connectors is intended to come within the scope of the present invention. The filtering system 14b is otherwise similar to the filtering system 14a.

A third tangential flow perfusion filtering system 14c is shown in FIG. 3C. The filtering system 14c replaces the connectors 38 and 40 with a second disposable aseptic connector 56. The filtering system 14c is otherwise similar to the filtering system 14a.

While the invention herein disclosed has been described by means of specific embodiments and applications thereof, numerous modifications and variations could be made thereto by those skilled in the art without departing from the scope of the invention set forth in the claims.

I claim:

1. A bioreactor filtering system comprising:
a bioreactor vessel containing bioreactor fluid;
a tangential flow filter in fluid communication with the bioreactor vessel;
a pump connected in series with the tangential flow filter for cycling the bioreactor fluid through the tangential flow filter; and
a pressure control valve controlling pressure in the tangential flow filter,
wherein the tangential flow filter, the pump and the pressure control valve are connected in series between a bioreactor outlet and bioreactor inlet receiving the bioreactor fluid from a bioreactor vessel and returning a filtered bioreactor fluid to the bioreactor vessel.

2. The bioreactor filtering system of claim 1, wherein the pump precedes the tangential flow filter and the tangential flow filter precedes the pressure control valve.

3. The bioreactor filtering system of claim 1, wherein the tangential flow filter is a hollow fiber tangential flow filter.

4. The bioreactor filtering system of claim 3, wherein hollow fiber tangential flow filter includes pore membranes greater than 0.1 micron which remove metabolic wastes and expressed proteins.

5. The bioreactor filtering system of claim 3, wherein hollow fiber tangential flow filter includes hollow fiber membranes with pore sizes less than 0.1 micron and can be utilized to generate a bioreactor environment which retains products of interest while passing metabolic wastes through the hollow fiber tangential flow filter.

6. The bioreactor filtering system of claim 1, wherein the pump is a low shear pump.

7. The bioreactor filtering system of claim 1, wherein the pump includes a disposable pumphead.

8. The bioreactor filtering system of claim 7, wherein the disposable pumphead is a low shear disposable pumphead.

9. The bioreactor filtering system of claim 8, wherein the disposable pumphead is a low shear gamma stable disposable pumphead.

10. The bioreactor filtering system of claim 7, wherein the disposable pumphead is a low shear disposable levitating pumphead,

11. The bioreactor filtering system of claim 1, wherein the pump and tangential flow filter are connected by flexible tubing allowing easy changing of the elements.

12. The bioreactor filtering system of claim 1, further including a permeate pump drawing metabolic waste and associated proteins from the hollow fiber tangential flow filter.

13. A bioreactor filtering system comprising:
a bioreactor vessel containing bioreactor fluid;
a hollow fiber tangential flow filter in fluid communication with the bioreactor vessel;
a pump in fluid communication with a bioreactor outlet of the bioreactor vessel and an inlet of the hollow fiber tangential flow filter, the pump including a low shear disposable pumphead;
a Transmembrane Pressure (TMP) pressure control valve connected serially between the hollow fiber tangential flow filter and a bioreactor inlet of the bioreactor vessel and controlling pressure in the hollow fiber tangential flow; and
a permeate pump drawing metabolic waste and associated proteins from the hollow fiber tangential flow filter, the waste and proteins separated from the bioreactor fluid by the hollow fiber tangential flow filter,
wherein the hollow fiber tangential flow filter, the pump, and the TMP pressure control valve are serially connected by flexible tubing between the bioreactor outlet and the bioreactor inlet receiving the bioreactor fluid from a bioreactor vessel and returning a filtered bioreactor fluid to the bioreactor vessel

14. A bioreactor filtering system comprising:
a bioreactor vessel containing bioreactor fluid;
a hollow fiber tangential flow filter in fluid communication with the bioreactor vessel;
a pump in fluid communication with a bioreactor outlet of the bioreactor vessel and an inlet of the hollow fiber tangential flow filter, the pump including a low shear gamma stable disposable pumphead including a magnetically levitated rotor;
a Transmembrane Pressure (TMP) pressure control valve connected serially between the hollow fiber tangential flow filter and a bioreactor inlet of the bioreactor vessel and controlling pressure in the hollow fiber tangential flow; and
a permeate pump drawing metabolic waste and associated proteins from the hollow fiber tangential flow filter, the waste and proteins separated from the bioreactor fluid by the hollow fiber tangential flow filter,
wherein the hollow fiber tangential flow filter, the pump, and the TMP pressure control valve are serially connected by flexible tubing between the bioreactor outlet and the bioreactor inlet receiving the bioreactor fluid from a bioreactor vessel and returning a filtered bioreactor fluid to the bioreactor vessel
